(19) United States
(12) Patent Application Publication
(10) Pub. No.: US 2025/0114768 A1
(43) Pub. Date: Apr. 10, 2025

(54) POROUS PROTEIN STRUCTURES

(71) Applicant: UWM Research Foundation, Inc., Milwaukee, WI (US)

(72) Inventor: Ionel Popa, Bayside, WI (US)

(21) Appl. No.: 18/833,831

(22) PCT Filed: Jan. 27, 2023
(86) PCT No.: PCT/US2023/061468

§ 371 Date: Jul. 26, 2024

Related U.S. Application Data
(60) Provisional application No. 63/304,212, filed on Jan. 28, 2022.

(51) Int. Cl. B01J 20/285; B01D 15/38; B01J 20/24; B01J 20/28; B01J 20/30; C07K 1/16; C07K 14/195; C07K 14/765

(52) U.S. Cl. CPC B01J 20/285; B01D 15/3809; B01J 20/24; B01J 20/28085; B01J 20/3057; C07K 1/16; C07K 14/195; C07K 14/765

(57) ABSTRACT

Disclosed herein are compositions that include a porous network of crosslinked protein having advantageous mechanical properties. An example composition includes a network of crosslinked protein, the network of crosslinked protein including a plurality of pores, each pore having a diameter of greater than 1 μm, wherein the network of crosslinked protein has a Young's modulus of greater than 1 kPa. Also disclosed herein are methods of making the compositions and methods of using the compositions, such as separating an analyte from a sample.

### CROSS-REFERENCE TO RELATED APPLICATIONS

This application claims priority to U.S. Provisional Patent Application No. 63/304,212 filed on Jan. 28, 2022, which is incorporated by reference herein in its entirety.

### TECHNICAL FIELD

This disclosure relates to porous compositions that include a network of crosslinked protein and their use in applications, such as purification and filtration methods.

### INTRODUCTION

Protein hydrogels are a class of material made from soluble proteins, which are covalently crosslinked and can be produced in a desired shape or size. Hydrogels made from globular proteins into a stable network are useful due to their applications in artificial tissue design, cell culture scaffolds, and as systems to study the mechanical and biochemical unfolding of proteins in crowded environments such as inside of cells. Due to their small size (e.g., typically 2-5 nm), the primary network allows only for a limited transfer of molecules and prevents the passing of particles and aggregates over 100 nm. Accordingly, protein-based materials with greater permeability, but without compromised mechanical properties, would be useful in a variety of applications.

### SUMMARY

In one aspect, disclosed are compositions including a network of crosslinked protein, the network of crosslinked protein including a plurality of pores, each pore having a diameter of greater than 500 nm, wherein the network of crosslinked protein has a Young's modulus of greater than 1 kPa.

In another aspect, disclosed are compositions including a network of crosslinked protein, the network of crosslinked protein including a plurality of pores, wherein the protein comprises an albumin, an antibody binding protein, an antibody, a fragment thereof, or a combination thereof, each pore has a diameter of greater than 1 μm, and the network of crosslinked protein has a Young's modulus of greater than 1 kPa and less than 100 kPa.

In another aspect, disclosed are methods of making a porous network of crosslinked protein, the method including: a. combining a protein and a polysaccharide in a solvent to provide a mixture; b. crosslinking the protein to provide a partial first network; c. crosslinking the partial first network and the polysaccharide, wherein crosslinking the partial first network provides a first network and crosslinking the polysaccharide provides a second network dispersed in the first network; and d. adding the first network and the second network to a solvent, wherein the solvent removes the second network from the first network to provide a porous network of crosslinked protein.

In another aspect, disclosed are methods of separating an analyte from a sample, the method including contacting the sample with a chromatography matrix, the chromatography matrix including a composition as disclosed herein; and separating the analyte from the sample.

### BRIEF DESCRIPTION OF THE DRAWINGS

FIG. 1 shows a schematic of an example method of providing porous polyprotein compositions. (Top): Representation of the gelation process of bovine serum albumin (BSA) molecules (represented as circles) while exposed to a light-activated reaction. (Bottom): Representation of an example approach to construct porous protein-based materials. Following a short exposure to light, which results in incomplete gelation, the material is later immersed in Ca²⁺, which can trigger nucleation and growth of alginate clusters. These alginate clusters can reshape the material network without significantly affecting the interactions between the protein multimers. The alginate is removed through diffusion in Tris or EDTA, leaving a porous structure.

FIG. 2A and FIG. 2B show scanning electron microscopy (SEM) images of cross-sections from BSA-alginate hydrogels. Schematics of the orientation during image acquisition (left images for FIG. 2A and FIG. 2B). Ensemble images (middle images for FIG. 2A and FIG. 2B) and zoom-ins (right images for FIG. 2A and FIG. 2B) for BSA-alginate samples before (FIG. 2A) and after (FIG. 2B) removal of alginate. BSA-alginate hydrogels maintained in CaCl₂ solution prior to imaging display a dense morphology with minimal pores (FIG. 2A). When immersed into EDTA or TRIS buffer, Ca²⁺ exposed BSA-alginate exhibit a homogeneous distribution of pores, left by the diffusion of the calcium-alginate network (FIG. 2B).

FIG. 3 shows confocal laser scanning microscope images of BSA and BSA-alginate hydrogels. (left image) A BSA hydrogel imaged after incubation in Tris buffer exhibits a dense morphology, lacking noticeable pores. (right image) A BSA-alginate hydrogel imaged after incubation in EDTA or TRIS buffer contains large, uniformly distributed pores, resulting from the vacancies left by the diffusion of the calcium-alginate network, with a diameter of 3±1 μm.

FIG. 4A, FIG. 4B, FIG. 4C, FIG. 4D, FIG. 4E, and FIG. 4F show mechanical behavior of BSA and BSA-alginate hydrogels. (FIG. 4A) Hydrogel samples are tethered between the hooks of a custom-made force-clamp rheometer and subjected to various force protocols. A PID-regulated feedback loop ensures that the applied force accurately follows the set force, while corresponding changes in sample length are recorded. (FIG. 4B) Stress-strain curves of BSA-alginate and BSA hydrogels in various buffer conditions are generated from force-clamp rheometer data. The Young's Modulus is calculated from the linear portion of the trace. (FIG. 4C) and (FIG. 4D) Release kinetics for the second alginate network from the BSA gel as a function of time. (FIG. 4C) Stress-strain curves of the same gel measured in 15 min intervals, and (FIG. 4D) the corresponding Young's moduli. (FIG. 4E) and (FIG. 4F) Effect of the primary network crosslinking time before inducing the formation of the secondary alginate network. (FIG. 4E) Stress-strain curves in Ca²⁺ and after 30 min immersion in Tris, and (FIG. 4F) the corresponding Young's moduli for Ca²⁺ (left bars) and Tris (right bars).

FIG. 5 shows BSA and BSA-alginate example filters. Example BSA and BSA-alginate hydrogels were fabricated in a disk shape and inserted into columns to serve as filters. The columns were loaded with (left) eYFP-vinculin (size ˜3-by-6 nm), (center) Limino Avocado Green dye (size ˜264 nm), and (right) Limino Cherry Red dye (size ˜290 nm). The samples were both centrifuged for 0 min (top panels), and 1 min (bottom panels), at 100 g centrifugal force. The left tube is less porous BSA, while the right tube is alginate-induced porous BSA. Due to the increased porosity, the alginate-induced porous BSA filters consistently passed the dye solution in less than 2 min (4 repetitions).

FIG. 6A and FIG. 6B show a diffusion model to describe the nucleation of alginate as a mechanism to produce porous protein gels. (FIG. 6A—top) 3D representation of three hydrogels made inside a symmetric box with a size of 150×150×150 nm³ and having 2 mM BSA and varying nucleation rates (I), shown above. (FIG. 6A—bottom) Cross-section of the gels in (FIG. 6A—top) showing the largest formed pore as a sphere. (FIG. 6B—top) Middle cross-section plane showing the protein molecules in white. The intensity changes with separation from the center plane. (FIG. 6B—bottom) Collapse image along the Z-plane of all the molecules inside the simulation volume. Pass-through pores are visible for Γ=100 and 1000.

FIG. 7 shows an estimation of pore dimensions for the diffusion model. (FIG. 7A) Cumulative probability distribution function (CPDF) of the particles inside a gel with 150 nm box size as a function of growth factor. Each simulation was repeated 5 times. The CPDF shifts capture the formation of the center pore. The distance representing two molecular diameters is indicated with an arrow. (FIG. 7B) Change in the diameter of the maximum pore size as a function of growth factor for the three box sizes considered. The 100 nm box size produces pores with smaller dimensions, due to the box constrains. The line represents a fit using a power law. The simulations were repeated 5 times for box sizes of 100 and 150 nm and 3 times for box size of 200 nm. Solid line represents fit of a power law dₚₒᵣₑ^{max}=19.9+1·Γ^{0.6} (nm).

FIG. 8 shows a SEM image of porous (protein L)₈ (e.g., 8 protein L molecules attached in tandem) networks polymerized in the presence of alginate after removal of the secondary alginate network.

FIG. 9A and FIG. 9B show column efficiency testing using example compositions of (protein L)₈. (FIG. 9A) Column made from 1.5 mM protein L8 and 0.5% (w/v) alginate, with the secondary reaction initiated after 10 min, crosslinked under light for 30 min. The lanes represent the molecular ladder, the pass through (showing non-adsorbed antibody), the final wash (W6) showing that all non-specifically adsorbed antibody was washed, the three elution steps in acid pH (E1, E2, E3), and a second molecular ladder. (FIG. 9B) Column made from pure protein L8, without alginate, of 1 mM concentration, crosslinked under light for 30 min. The lanes represent the molecular ladder, the pass through (showing non-adsorbed antibody), the final wash (W6) showing that all non-specifically adsorbed antibody was washed, the three elution steps in acid pH (E1, E2, E3), and a second molecular ladder.

FIG. 10 shows a schematic of an example column-based purification method using the disclosed compositions.

### DETAILED DESCRIPTION

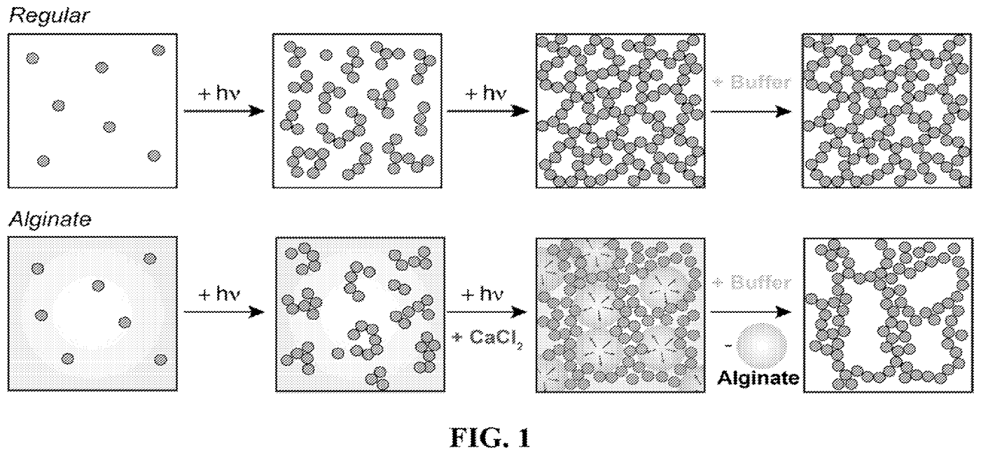

Disclosed herein are methods that can produce porous materials by fine-tuning two competing cross-linking reactions, one generating a dissolvable physical backbone and the other resulting in a covalently-linked chemical network (FIG. 1). Unlike other approaches used to induce pores inside biomaterials, the disclosed method does so without compromising the mechanical properties of the primary network. The permeability of the protein-based materials with micrometer-sized pores was tested by producing cylindrical shapes that can accommodate regular centrifugation columns and testing the passing of a small protein and of two dyes. In all cases, the alginate-induced porous protein materials had a superior permeability. In addition, a diffusion model was used to describe the formation of pores through the dynamic competition between cross-linking of protein molecules and protein-clusters and the nucleation of polysaccharide into nucleation centers. The disclosed methods and porous compositions provided therefrom can be applied in a variety of applications, such as studying protein-protein interactions, cell growth, and designing affinity methods.

### 1. DEFINITIONS

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, the term “analyte” refers to a substance that is of interest, e.g., of being identified, characterized, measured, etc., through an analytical procedure or technique. The analyte can be a “biomarker,” which refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, a cancer cell, etc.), small molecules, lipids, vesicles, carbohydrates, nucleic acids, DNA, RNA, peptides, proteins, enzymes, antigens, and antibodies. A biomarker can be derived from an infectious agent, such as a bacterium, fungus or virus, or can be an endogenous molecule that is found in greater or lesser abundance in a subject suffering from a disease or disorder as compared to a healthy individual (e.g., an increase or decrease in expression of a gene or gene product).

A “protein” or “polypeptide” is a linked sequence of 50 or more amino acids linked by peptide bonds. A peptide is a linked sequence of 2 to 50 amino acids linked by peptide bonds. The polypeptide and peptide can be natural, synthetic, or a modification or combination of natural and synthetic. Proteins and polypeptides can include proteins such as binding proteins, receptors, and antibodies. The terms “polypeptide,” and “protein” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tall domains, “Domains” are portions of a polypeptide that form a compact unit of the polypeptide and are typically 15 to 350 amino acids long. Example domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length, in some embodiments, a motif includes 3, 4, 5, 6, or 7 sequential amino acids. A domain may be comprised of a series of motifs, which may be similar or different.

“Sample” or “test sample,” as used herein, refers to any sample in which the presence and/or level of an analyte is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “variant,” as used herein, refers to a peptide or protein that differs in amino acid sequence by the insertion, deletion, or conservative substitution of amino acids, but retain at least one biological activity relative to a reference peptide or protein. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids (see Kyte et al., J. Mol. Biol. 1982, 757, 105-132, which is incorporated by reference herein in its entirety). The hydropathic index of an amino acid is based on a consideration of its hydrophobicity and charge. It is known in the art that amino acids of similar hydropathic indexes can be substituted and retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide. Substitution of amino acids having similar hydrophilicity values can result in polypeptides retaining biological activity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITIONS

Disclosed herein are compositions that include a network of crosslinked protein. The crosslinked protein has a porous structure that includes a plurality of pores. In addition, despite its porous structure, the network of crosslinked protein can still have advantageous mechanical properties, such as a similar Young's modulus as a non-porous protein-based hydrogel, which can be useful in overall stability of the network and composition thereof.

The composition may include further components depending on the application of the composition. For example, the composition may further include a binding ligand. A binding ligand, as used herein, refers to a molecule that can specifically interact or specifically bind with a target molecule, such as an analyte of interest. The binding ligand can, e.g., be used to aid the separation of an analyte from a sample in the methods disclosed herein. Example binding ligands include, but are not limited to, aptamers, carbohydrates, proteins, antibodies, single chain variable fragments, and the like. In some embodiments, the binding ligand is a protein. In some embodiments, the binding ligand is an antibody.

The binding ligand can be associated with the network of crosslinked protein. For example, the binding ligand can be physically contacting the network of crosslinked protein, such as being adsorbed or adhered to the network of crosslinked protein. In some embodiments, the binding ligand is associated with the network of crosslinked protein by being attached through, e.g., electrostatic or covalent interactions with the crosslinked protein. In some embodiments, the protein includes the binding ligand. For example, the network of crosslinked protein can include crosslinked binding ligand. Or in other words, the protein used to provide the crosslinked protein can be a binding ligand, such as an antibody or an antibody binding protein.

### A. Networks of Crosslinked Protein

As used herein, a network of crosslinked protein refers to a plurality of protein molecules that are interconnected through crosslinks. The description that follows for the protein can also be applied to individual protein molecules when applicable. As discussed elsewhere, crosslinking the protein via methods disclosed herein can provide a network of crosslinked protein that has a porous structure. Accordingly, the network of crosslinked protein includes a plurality of pores.

Because of the methods disclosed herein, the network of crosslinked protein can include pores having a diameter greater than typically seen with porous protein-based structures. For example, the network of crosslinked protein can include a plurality of pores, where each pore can have a diameter of greater than 500 nm, greater than 600 nm, greater than 700 nm, greater than 800 nm, greater than 900 nm, greater than 1 μm, greater than 1.5 μm, greater than 2.5 μm, greater than 3 μm, greater than 3.5 μm, greater than 4 μm, greater than 4.5 μm, or greater than 5 μm. In some embodiments, the network of crosslinked protein includes a plurality of pores, each pore having a diameter of less than 12 μm, less than 10 μm, less than 9.5 μm, less than 9 μm, less than 8.5 μm, less than 8 μm, less than 7.5 μm, less than 7 μm, less than 6.5 μm, or less than 6 μm. In some embodiments, the network of crosslinked protein includes a plurality of pores, each pore having a diameter of about 500 nm to about 10 μm, such as about 700 nm to about 10 μm, about 900 nm to about 10 μm, about 500 nm to about 5 μm, about 900 nm to about 6 μm, about 1 μm to about 10 μm, about 1.5 μm to about 9.5 μm, about 2 μm to about 8 μm, about 2 μm to about 7 μm, about 4 μm to about 10 μm, about 5 μm to about 10 μm, about 1 μm to about 6 μm, or about 1 μm to about 5 μm. The network of crosslinked protein may also include pore(s) that are smaller in diameter than 1 μm. Pore size can be measured by techniques known within the art, such as, but not limited to, electron microscopy (e.g., SEM) and confocal laser scanning microscopy.

The type of protein used to provide the crosslinked network is not generally limited and can include any type of protein that can be crosslinked as disclosed herein. Example proteins include, but are not limited to, albumin, antibody binding proteins, antibodies, and fragments thereof. Examples of antibody binding proteins include, but are not limited to, protein L, protein M, protein G, and protein A. Examples of albumin can be from any number of species including, but not limited to, human, mouse, and bovine. As mentioned above, the protein can be a plurality of protein molecules. The protein can also include one type of protein or can be 2, 3, 4, 5, or more different types of proteins. For example, the protein may include both albumin and an antibody. In addition, the protein can include a protein that is a plurality of proteins attached, e.g., covalently, in tandem (prior to crosslinking). For example, the protein can include a protein that is 2 to 20 proteins attached in tandem, such as 2 to 18, 3 to 15, 4 to 16, or 3 to 12 proteins attached in tandem. In some embodiments, the protein includes a protein that is 8 protein L molecules attached in tandem. Attaching a plurality of proteins in tandem can be beneficial to the crosslinking process, the final properties of the network, or both.

In some embodiments, the protein includes albumin, protein L, protein M, protein G, protein A, an antibody, or a combination thereof. In some embodiments, the protein includes an amino acid sequence of SEQ ID NO: 1, SEQ ID NO: 2, SEQ ID NO: 3, SEQ ID NO: 4, or a combination thereof. In some embodiments, the protein includes an amino acid sequence of SEQ ID NO: 1, SEQ ID NO: 2, SEQ ID NO: 3, or SEQ ID NO: 4.

The protein can also have a certain amount of tyrosine residues, where this amino acid residue can be beneficial for crosslinking reactions. In some embodiments, the protein includes at least 3 tyrosine residues, at least 4 tyrosine residues, at least 5 tyrosine residues, at least 6 tyrosine residues, at least 7 tyrosine residues, at least 8 tyrosine residues, at least 9 tyrosine residues, at least 10 tyrosine residues, at least 11 tyrosine residues, at least 12 tyrosine residues, at least 13 tyrosine residues, at least 14 tyrosine residues, at least 15 tyrosine residues, at least 16 tyrosine residues, at least 17 tyrosine residues, at least 18 tyrosine residues, at least 19 tyrosine residues, at least 20 tyrosine residues, at least 25 tyrosine residues, at least 30 tyrosine residues, or at least 35 tyrosine residues. In some embodiments, the protein includes less than 50 tyrosine residues, less than 45 tyrosine residues, less than 40 tyrosine residues, less than 35 tyrosine residues, less than 30 tyrosine residues, less than 25 tyrosine residues, less than 20 tyrosine residues, less than 15 tyrosine residues, less than 10 tyrosine residues, or less than 5 tyrosine residues. In some embodiments, the protein includes 3 to 50 tyrosine residues, such as 3 to 20, 5 to 50, or 3 to 40.

The protein, prior to crosslinking, can have a varying molecular weight. For example, the protein can have a molecular weight of about 20 kilodaltons (kDa) to about 100 kDa, such as about 25 kDa to about 95 kDa, about 30 kDa to about 80 kDa, about 20 kDa to about 95 kDa, about 20 kDa to about 80 kDa, about 30 kDa to about 100 kDa, or about 40 kDa to about 100 kDa. In some embodiments, the protein has a molecular weight of less than 100 kDa, less than 95 kDa, less than 90 kDa, less than 85 kDa, less than 80 kDa, or less than 75 kDa. In some embodiments, the protein has a molecular weight of greater than 20 kDa, greater than 25 kDa, greater than 30 kDa, greater than 35 kDa, greater than 40 kDa, or greater than 45 kDa. Molecular weight of the protein can be measured by techniques known within the art, such as, but not limited to, liquid chromatography, mass spectrometry, and sodium dodecyl sulfate-polyacrylamide gel electrophoresis (SDS-PAGE) analysis.

As discussed in more detail below, the protein can be crosslinked in a number of different ways known within the art. For example, the protein can be crosslinked by enzymatic crosslinking, photocrosslinking, thermal crosslinking, a crosslinking agent, or a combination thereof. In some embodiments, the protein is crosslinked by enzymatic crosslinking, photocrosslinking, thermal crosslinking, or a crosslinking agent. In some embodiments, the protein is crosslinked by photocrosslinking.

The network of crosslinked protein has a number of beneficial properties that make it useful in a variety of applications. For example, the network of crosslinked protein can have a Young's modulus of about 1 kPa to about 200 kPa, such as about 1 kPa to about 150 kPa, about 1 kPa to about 100 kPa, about 1 kPa to about 90 kPa, about 1 kPa to about 80 kPa, about 1 kPa to about 70 kPa, about 1 kPa to about 60 kPa, about 1 kPa to about 50 kPa, about 1 kPa to about 40 kPa, about 1 kPa to about 30 kPa, about 1 kPa to about 20 kPa, about 5 kPa to about 100 kPa, about 5 kPa to about 50 kPa, about 5 kPa to about 25 kPa, about 5 kPa to about 20 kPa, about 10 kPa to about 30 kPa, or about 10 kPa to about 25 kPa. In some embodiments, the network of crosslinked protein has a Young's modulus of greater than 1 kPa and less than 100 kPa, such as greater than 2 kPa and less than 75 kPa, greater than 3 kPa and less than 60 kPa, greater than 4 kPa and less than 50 kPa, greater than 5 kPa and less than 40 kPa, or greater than 1 kPa and less than 50 kPa. In some embodiments, the network of crosslinked protein has a Young's modulus of greater than 1 kPa, greater than 2 kPa, greater than 3 kPa, greater than 4 kPa, greater than 5 kPa, greater than 6 kPa, greater than 7 kPa, greater than 8 kPa, greater than 9 kPa, greater than 10 kPa, greater than 11 kPa, greater than 12 kPa, greater than 13 kPa, greater than 14 kPa, or greater than 15 kPa. In some embodiments, the network of crosslinked protein has a Young's modulus of less than 200 kPa, less than 150 kPa, less than 110 kPa, less than 100 kPa, less than 90 kPa, less than 80 kPa, less than 70 kPa, less than 60 kPa, less than 50 kPa, less than 40 kPa, less than 30 kPa, or less than 25 kPa. The Young's modulus of the network of crosslinked protein can be measured by techniques known within the art, such as, but not limited to, rheometers (e.g., force-clamp rheometer).

In some embodiments, the network of crosslinked protein is a hydrogel.

In some embodiments, the composition includes a network of crosslinked protein, the network of crosslinked protein including a plurality of pores, each pore having a diameter of greater than 500 nm, wherein the network of crosslinked protein has a Young's modulus of greater than 1 kPa.

In some embodiments, the composition includes a network of crosslinked protein, the network of crosslinked protein including a plurality of pores, wherein the protein includes an albumin, an antibody binding protein, an antibody, a fragment thereof, or a combination thereof, each pore has a diameter of greater than 1 μm, and the network of crosslinked protein has a Young's modulus of greater than 1 kPa and less than 100 kPa.

### 3. METHODS

### A. Methods of Making a Porous Network of Crosslinked Protein

The present disclosure also relates to methods of making porous networks of crosslinked protein that can have advantageous physical properties, such as a beneficial Young's modulus. The method can include combining a protein and a polysaccharide in a solvent to provide a mixture (e.g., step a). Description of the protein above can also be applied to the methods of making a porous network. Examples of polysaccharides include, but are not limited to, alginate, cellulose (e.g., cellulose microfibrils), carrageenan, pectin, sulfated polysaccharides, and combinations thereof. Further discussion on polysaccharides and multivalent crosslinking can be found in Wurm et al., Multivalent Ions as Reactive Crosslinkers for Biopolymers-A Review, Molecules. 2020 April; 25(8): 1840, which is incorporated by reference herein in its entirety. In some embodiments, the polysaccharide includes alginate. In some embodiments, the polysaccharide is alginate. In some embodiments, the protein includes an albumin, an antibody binding protein, an antibody, a fragment thereof, or a combination thereof, and the polysaccharide includes alginate.

The polysaccharide, prior to crosslinking, can have a varying molecular weight. For example, the polysaccharide can have a molecular weight of about 10 kDa to about 100 kDa, such as about 20 kDa to about 95 kDa, about 30 kDa to about 80 kDa, about 20 kDa to about 95 kDa, about 20 kDa to about 80 kDa, about 30 kDa to about 100 kDa, or about 40 kDa to about 100 kDa. In some embodiments, the polysaccharide has a molecular weight of less than 100 kDa, less than 95 kDa, less than 90 kDa, less than 85 kDa, less than 80 kDa, or less than 75 kDa. In some embodiments, the protein has a molecular weight of greater than 10 kDa, greater than 20 kDa, greater than 30 kDa, greater than 35 kDa, greater than 40 kDa, or greater than 45 kDa. Molecular weight of the polysaccharide can be measured by techniques known within the art, such as, but not limited to, liquid chromatography and mass spectrometry.

The solvent is generally not limited as long as it can dissolve the protein and the polysaccharide. Example solvents include, but are not limited to, aqueous buffers (e.g., Tris, PBS, and Hepes, optionally with about 150 mM monovalent salt). The polysaccharide can be added to the solvent prior to the protein, after the protein, or at the same time.

The protein and the polysaccharide can be included in the mixture in varying amounts. For example, the protein can be included in the mixture at about 0.5 mM to about 5 mM, such as about 0.75 mM to about 4 mM, about 1 mM to about 3 mM, or about 1.5 mM to about 2.5 mM. In addition, the polysaccharide can be included in the mixture at about 0.5% (w/v) to about 5% (w/v), such as about 0.75% (w/v) to about 4% (w/v), about 1% (w/v) to about 3% (w/v), or about 1.5% (w/v) to about 2.5% (w/v).

The method can include crosslinking the protein to provide a partial first network (e.g., step b). The protein can be partially crosslinked without crosslinking the polysaccharide. Partially crosslinked refers to a network of crosslinked protein that has not completely finished the crosslinking reaction. For example, the partial first network can be no more than 1%, no more than 5%, no more than 10%, no more than 15%, no more than 20%, no more than 25%, no more than 30%, no more than 35%, no more than 40%, no more than 45%, or no more than 50% crosslinked compared to the first network provided in step c, where the first network is considered a completed crosslinking reaction (e.g., 100% crosslinked). In some embodiments, the partial first network is crosslinked about 0.1% to about 50% compared to the first network, such as about 0.5% to about 50%, about 1% to about 50%, about 5% to about 50%, about 1% to about 40%, about 1% to about 30%, about 1% to about 20%, about 1% to about 40%, about 2% to about 40%, about 3% to about 30%, about 10% to about 50%, about 15% to about 50%, about 20% to about 50%, or about 0.5% to about 20% compared to the first network.

The protein can be crosslinked for a varying amount of time to provide the partial first network. For example, crosslinking the protein can be performed from about 1 second to about 10 minutes, such as about 5 seconds to about 10 minutes, about 30 seconds to about 10 minutes, about 1 minute to about 10 minutes, about 1 second to about 7 minutes, about 5 seconds to about 7 minutes, about 1 second to about 5 minutes, about 10 seconds to about 6 minutes, or about 10 seconds to about 5 minutes. In some embodiments, crosslinking the protein to provide the partial first network is performed no more than 10 minutes, no more than 9 minutes, no more than 8 minutes, no more than 7 minutes, no more than 6 minutes, or no more than 5 minutes.

As mentioned above, the crosslinking reaction can be performed in a number of different ways as known within the art. For example, the protein can be crosslinked by enzymatic crosslinking, photocrosslinking, thermal crosslinking, a crosslinking agent, or a combination thereof. In some embodiments, the protein is crosslinked by enzymatic crosslinking, photocrosslinking, thermal crosslinking, or a crosslinking agent. Example crosslinking agents include, but are not limited to, light activated crosslinking agents, redox-activated crosslinking agents, or a combination thereof. In some embodiments, the crosslinking reaction does not include a crosslinking agent. In some embodiments, the first network, the partial first network, or both are covalently crosslinked.

The protein can be crosslinked by photocrosslinking. For example, the protein can be covalently crosslinked through a reaction triggered by exposure to light (e.g., via a mercury lamp). The photocrosslinking reaction can include a crosslinking reagent(s) that can initiate the crosslinking reaction after exposure to light. Example reagents includes, but are not limited to, ammonium persulfate (APS) and tris(bypyridine) ruthenium(II) chloride [Ru(bpy)₃]²⁺. Photocrosslinking reactions can crosslink the protein(s) through different amino acids. For example, the protein can be crosslinked through tyrosine residues.

The method can further include crosslinking the partial first network and the polysaccharide, wherein crosslinking the partial first network provides a first network and crosslinking the polysaccharide provides a second network dispersed in the first network (e.g., step c). The first network can also be referred to as a network of crosslinked protein and the second network can also be referred to as a network of crosslinked polysaccharide. Crosslinking the partial first network and the polysaccharide can include extruding the partial first network and the polysaccharide into a container that initiates crosslinking the polysaccharide (e.g., divalent cation solution) and the partial first network can also have its crosslinking reaction continued. The crosslinking of the partial first network and the polysaccharide can take place at the same time (e.g., simultaneously). In addition, the second network can form spherical structures dispersed in the first network. The second network can also be homogeneously dispersed in the first network.

The partial first network and the polysaccharide can each independently be crosslinked for a varying amount of time to provide the first network and the second network. For example, crosslinking the partial first network and the polysaccharide can each independently be performed for about 1 minute to about 30 minutes, such as about 5 minutes to about 25 minutes, about 10 minutes to about 20 minutes, about 1 minute to about 25 minutes, about 10 minutes to about 30 minutes, or about 5 minutes to about 20 minutes. In some embodiments, crosslinking the partial first network and the polysaccharide can each independently be performed for no more than 10 minutes, no more than 15 minutes, no more than 20 minutes, no more than 25 minutes, or no more than 30 minutes.

The polysaccharide can be crosslinked by techniques as described above for the protein, as along as the crosslinked polysaccharide can be removed from the network of crosslinked protein. In addition to those techniques, the polysaccharide can be crosslinked via electrostatic crosslinking, e.g., through multivalent cations. In some embodiments, the polysaccharide is crosslinked via divalent cations. In some embodiments, the polysaccharide is crosslinked via Ca²⁺. In some embodiments, the first network is covalently crosslinked and the second network is electrostatically crosslinked.

The method can also include adding the first network and the second network to a solvent, wherein the solvent removes the second network from the first network to provide a porous network of crosslinked protein (e.g., step d). The solvent can either dissolve the second network or remove the crosslinks of the second network. Removal of the second network can provide pores in the first network, and thus can provide a porous network of crosslinked protein. There may be some residual second network that remains in the first network following addition to the solvent (e.g., less than 5%, less than 4%, less than 3%, less than 2%, less than 1%, less than 0.9%, less than 0.5%, less than 0.1%, or less than 0.09% based on the weight of the composition). In addition, the solvent is generally not limited as long as it can dissolve the polysaccharide and/or network thereof. Example solvents include, but are not limited to, aqueous buffers (e.g., Tris buffer and EDTA). In some embodiments, the solvent of step d is the same solvent as step a.

In some embodiments, step a, step b, step c, and step d are done sequentially.

The description of the composition and the network of crosslinked protein can also be applied to the methods of making a porous network.

B. Methods of Separating an Analyte from a Sample

Also disclosed herein are methods of using the disclosed compositions for separating an analyte from a sample. The method can include contacting a sample with a chromatography matrix. As used herein, chromatography matrix refers to a solid phase used in chromatography, such as used within a chromatography column. Example chromatography columns include, but are not limited to, liquid chromatography columns, reversed phase chromatography columns, spin columns, and gravity-flow columns. The column can include a composition as disclosed herein. In some embodiments, the chromatography matrix consists essentially of a composition as disclosed herein. In some embodiments, the chromatography matrix consists of a composition as disclosed herein.

The analyte and sample are generally not limited within the disclosed methods. And, due to its advantageous pore size, the disclosed compositions and chromatography matrices thereof are capable of passing through molecules having a hydrodynamic diameter of greater than 75 nm, greater than 90 nm, greater than 100 nm, greater than 105 nm, greater than 110 nm, greater than 120 nm, greater than 150 nm, or greater than 200 nm. The hydrodynamic diameter of a molecule can be measured via dynamic light scattering. In some embodiments the disclosed compositions and chromatography matrices thereof are capable of passing through molecules having a hydrodynamic diameter of greater than 100 nm. In some embodiments, the analyte includes a protein, a carbohydrate, a lipid, a nucleic acid, or a combination thereof. In some embodiments, the analyte includes a protein. In some embodiments, the analyte is a protein. In some embodiments, the analyte is an antibody. In some embodiments, the analyte is a biomarker.

The method can include flowing a mobile phase through the chromatography matrix. Example mobile phases include, but are not limited to, aqueous phases, organic phases, or a combination thereof. The method can further include separating the analyte from the sample. The description of the composition and the networks of crosslinked protein can also be applied to the methods of separating an analyte.

The disclosed invention has multiple aspects, illustrated by the following non-limiting examples.

### 4. EXAMPLES

### Example 1

### Materials & Methods

Materials. Bovine Serum Albumin (BSA) was purchased from Rocky Mountain Biologicals. Sodium alginate and calcium chloride (CaCl₂) were purchased from Willpowder. Sodium phosphate monobasic anhydrous (NaH₂PO₄) was obtained from Research Products International. Sodium chloride (NaCl) and Rhodamine B were both purchased from Thermo Fisher Scientific. Ammonium persulfate (APS), tris(bypyridine) ruthenium(II) chloride [Ru(bpy)₃]²⁺, Trizma base, hydrochloric acid (37%), potassium hydroxide, and Sigmacote were purchased from Sigma-Aldrich. Tris [20 mM Tris-NaCl and 150 mM NaCl (PH ˜7.4)] and phosphate-buffered saline [10 mM NaH₂PO₄ and 150 mM NaCl (PH ˜7.4)] were used as buffers. APS and [Ru(bpy)₃]²⁺ were dissolved in Tris buffer to prepare stock solutions with respective concentrations of 1 M and 6.67 mM. For staining of gels, a 1 μg/mL solution of Rhodamine B in PBS buffer was used. A 5% (w/v) CaCl₂ solution (pH ˜7.4) was prepared for alginate reaction. Cherry Red and Avocado Green food dyes were purchased from Limino. eYFP fluorophore conjugated to vinculin head was expressed and purified following standard procedures (see Dahal, N et al., Binding-Induced Stabilization Measured on the Same Molecular Protein Substrate Using Single-Molecule Magnetic Tweezers and Heterocovalent Attachments. J Phys Chem B 2020, 124 (16), 3283-3290, which is incorporated by reference herein in its entirety). All solutions were prepared with double-distilled water (ddH₂O).

Synthesis of Protein-based Materials. Two varieties of protein-based hydrogels were synthesized: one containing only BSA (pure-protein) and the other containing BSA and alginate (protein-polysaccharide). For the BSA-alginate hydrogels, a 2 mM BSA 2% alginate (w/v) solution was prepared by first dissolving the alginate in a stock solution of Tris buffer to reach a concentration of 2% (w/v) sodium alginate and then dissolving the appropriate mass of BSA. Following dilution, the proteins were transformed into materials by mixing with the stock solutions of APS 1M and Rubpy 6.67 mM in a volume ratio of 15:1:1. To form cylindrical hydrogels, polytetrafluoroethylene (PTFE) tubes (inner diameter, 0.56 mm; Cole-Parmer) were passivated with Sigmacote for about 5 minutes and dried prior to loading mixture. BSA hydrogels were synthesized by placing the loaded tube in front of a 100 W mercury lamp for 30 minutes, allowing for complete gelation via the photoactivated formation of dityrosine covalent bonds. The lamp was placed about 20 cm away from the sample, to avoid thermal effects to the gel, and had a long pass filter of 400 nm, to prevent non-specific reactions from UV radiation. The sample was then extruded into Tris solution using the tip of a blunted needle. BSA-alginate hydrogels were prepared in a similar manner: the loaded PTFE tube was first placed in front of the 100 W mercury lamp for a variable amount of time (from 30 seconds to 30 minutes) to allow partial formation of the covalent dityrosine network. The sample was then extruded into a dish of 5% (w/v) CaCl₂ solution (pH ˜7.4) and placed in front of the mercury lamp for the remainder of 30 minutes, allowing simultaneous formation of the ionic calcium-alginate network within the covalent dityrosine network. The cylindrically shaped hydrogels were then used for rheometry measurements, SEM, and confocal microscopy imaging. For confocal microscopy imaging and filtration experiments, disk shaped hydrogels were also prepared by using a cylindrical mold with a diameter of 7.80 mm diameter and a height of ˜3 mm. The selected mold was passivated by applying Sigmacote for 5 minutes, and then dried before loading with an appropriate volume of mixture. BSA hydrogels were synthesized by placing the loaded mold in front of the 100 W mercury lamp for 30 minutes with a glass coverslip covering the mold, to prevent evaporation. After complete gelation, a blunted needle was used to carefully remove the disk-shaped sample from the mold and transfer it to Tris buffer solution. BSA-alginate hydrogels were synthesized by placing the loaded mold in front of a 100 W mercury lamp for 1 minute with a glass coverslip to reduce evaporation. Next, a blunted needle was used to carefully extrude the disk-shaped sample into a dish of 5% (w/v) CaCl₂ solution, which was placed in front of the mercury lamp for the remainder of 30 minutes. To form pores, the samples were initially exposed in EDTA. However, it was noticed that simple exposure to Tris buffer, without Ca²⁺ ions had a similar effect, and thus the EDTA step can be optional. As such, the measurements with EDTA and Tris washes were aggregated.

Mechanical Studies. A custom-made force-clamp rheometer was used for the mechanical characterization of hydrogel samples, as reported in previous studies (see Khoury, L. R. et al., Study of Biomechanical Properties of Protein-Based Hydrogels Using Force-Clamp Rheometry. Macromolecules 2018, 51 (4), 1441-1452 and Khoury, L. R et al., Force-Clamp Rheometry for Characterizing Protein-based Hydrogels. Jove-J Vis Exp 2018, (138)—both of which are incorporated by reference herein in their entirety). A cylindrical hydrogel sample is tethered between two hooks-one attached to a force sensor and the other to a voice coil motor—and a PID-regulated feedback loop ensures that the applied force matches the set point, as the sample is subjected to various force protocols. The force and corresponding gel extension data are used to generate stress-strain curves, the linear part of which reports Young's Modulus. Cylindrical hydrogel samples were immersed in room temperature solutions and then subjected to force ramps consisting of extension to a peak force at a rate of 40 Pa/s, relaxation to zero force at an equal rate, and constant zero force. The Young's modulus of pure-protein BSA hydrogels was measured in Tris buffer, serving as a control measurement. The Young's modulus of the BSA-Alginate hydrogels was measured first in 5% (w/v) CaCl₂, and then every 15 minutes over the course of one hour, following transfer to Tris buffer.

Scanning Electron Microscope (SEM) Imaging. SEM imaging was performed using a HITACHI S-4800 instrument. Cylindrical hydrogel samples made from 2 mM BSA dissolved in 2% (w/v) alginate solution were prepared. Following light exposure, one sample was maintained in 5% (w/v) CaCl₂ solution, and the other was washed in Tris or EDTA solution for at least 30 minutes to allow the removal of the calcium-alginate ionic network. The samples were then frozen in liquid nitrogen and lyophilized overnight. Dried samples were broken with forceps to expose the interior cross-sectional area and mounted on aluminum stubs with double sided carbon tape. The dye samples were similarly prepared by depositing a diluted solution on a mica slide. The samples were then left to dry in a vacuum chamber. All samples were coated with a 3 nm iridium conductive layer, using a sputter coater and imaged at an acceleration voltage of 5 keV.

Confocal Laser Scanning Microscope Imaging. Confocal laser scanning microscopy imaging was performed to compare the microstructural morphology of BSA and BSA-alginate hydrogels in Tris buffer solution. All images were taken using a Nikon CS2 scanning confocal microscope and Nikon NIS-Elements software. Cylindrical and disk-shaped hydrogels were prepared and then incubated in Tris buffer solution. The BSA-alginate samples were allowed to equilibrate to Tris solution for at least one hour to allow the calcium-alginate network to dissolve. The hydrogel samples were then stained in a 0.001 mg/mL Rhodamine B solution overnight and rinsed in PBS buffer for about 3 hours prior to imaging. Samples were cut to reveal the cross-sectional surface and mounted on a glass slide in PBS buffer. Imaging was conducted using a 40× water immersion lens and Z-stack images were taken at equal intervals. The images were analyzed using a custom-written program in Igor Pro (Wavemetrics).

Filtration. Disk shaped 2 mM BSA and 2 mM BSA 2% (w/v) alginate hydrogels were synthesized in a cylindrical mold (7.80 mm diameter, ˜3 mm height) as described above. Following synthesis, the disk shaped samples were loaded into plastic centrifuge columns to serve as filters and compacted with a plastic ring to prevent leakage. Both columns were first loaded with Tris buffer solution for 30 minutes, allowing the calcium-alginate network to dissolve from the 2 mM BSA 2% (w/v) alginate hydrogel.

The relative ability of the protein and protein-polysaccharide filters to allow various solution to pass was compared using Tris buffer, a solution of eYFP-vinculin, and diluted solutions of Cherry Red and Avocado Green food dyes. Prior to testing with each solution, both columns were washed with Tris buffer and ddH₂O and then dried. Next, each column was loaded with the same volume of solution and the mass of the loaded solution was recorded. A centrifuge was used to spin the samples at a speed of 1000 rpm until most solution had passed through either one or both filters. The mass of the filtrate that passed through each filter was measured at 1-5 minute intervals during the experiment to determine the rate of filtration. The pass-through concentrations were measured using a spectrophotometer (Shimadzu Diagnostics, BioSpec-Nano).

Gelation Simulations. A diffusion and cross-linking model of BSA hydrogels in the presence of alginate nucleation was developed using a previously described procedure for gelation of polyproteins (see Shmilovich, K et al., Modeling Protein-Based Hydrogels under Force. Phys Rev Lett 2018, 121 (16), 168101, which is incorporated by reference herein in its entirety). First, non-overlapping hard spheres of 3.5 nm radius, which is the hydrodynamic radius measured for BSA, were placed in a cubic volume of 50, 100 or 200 nm. The number of molecules was calculated to correspond to a BSA concentration of 2 mM (volume fraction 0.216). The placement was done by growing randomly placed points within the volume element to spheres with R=3.5 nm, and repositioning the overlapping molecules at every step. This configuration resembles the BSA molecules fully dissolved in solution, which do not aggregate at the considered concentration. The molecules were then left to diffuse and connections were made when two spheres came within a distance smaller than twice the protein radius. The parameters corresponding to the diffusion of clustered molecules were calculated from their radius of gyration R_{g}, which, for N_{d} molecules forming a cluster, depends on the root-mean-square distance of the segments of the molecule rᵢ from its center of mass R_{COM}.

$${R}_{g}^{2} = \frac{1}{{N}_{d}} {\sum }_{i = 1}^{{N}_{d}} {\vert {R}_{COM} - {r}_{i} \vert}^{2}$$

and were for translation (t) and rotation (θ) estimated as:

$${D}_{{t}_{clust}} = \frac{{k}_{B} T}{6 πη {R}_{g}} \\ {D}_{{θ}_{clust}} = \frac{{k}_{B} T}{8 πη {R}_{g}^{3}}$$

where η is the dynamic viscosity of water, and k_{B}T is the thermal energy.

To simulate nucleation and growth of alginate, the growth rate was considered as a repulsive force field from the center of the simulation volume, that acts on the molecules and clusters with an intensity inversely proportional with their location from the center:

$${F}_{n} = \frac{Γ}{{( {r}_{i}^{2} )}^{1 / 2}} χ$$

where Γ represents the nucleation growth factor for the alginate clusters, and χ is the ratio between the radius of a particle and of the cluster (equal to 1 for a single particle). During diffusion, a ‘soft’ spherical boundary was used, where molecules crossing the simulation volume had their orientation vector repositioned to point inwards. All simulations were run for a total of 100 ns. All the parameters used in the simulations. Maximum pore size was evaluated using the maximum ball approach and cumulative probability distribution function (CPDF) between molecules (see Tyagi, M et al., Probability density function approach for modelling multi-phase flow with ganglia in porous media. J Fluid Mech 2011, 688, 219-257, which is incorporated by reference herein in its entirety).

### Example 2

### Porous Protein Structures—BSA

Protein-based materials were prepared using a photoactivated reaction involving tris(bipyridine) ruthenium(II) chloride ([Ru(bpy)₃]²⁺) to covalently link exposed Tyrosine amino acids on adjacent molecules of BSA proteins. The resulting dityrosine network produces a gel with mechanical properties that can be fine-tuned according to the concentration of protein used. BSA hydrogels have a Young's Modulus ranging from 2.6 to 16 kPa for BSA hydrogels ranging in concentration from 1 to 4 mM (see Khoury, L. R, et al., Study of Biomechanical Properties of Protein-Based Hydrogels Using Force-Clamp Rheometry. Macromolecules 2018, 51 (4), 1441-1452, which is incorporated by reference herein in its entirety). A secondary ionic network can then be formed through the association of alginate chains in the presence of Ca²⁺ cations.

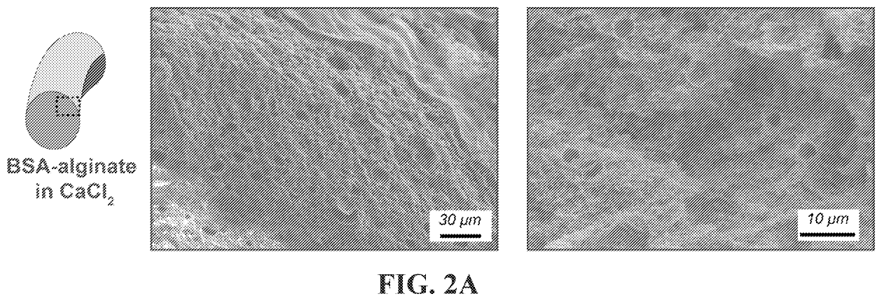

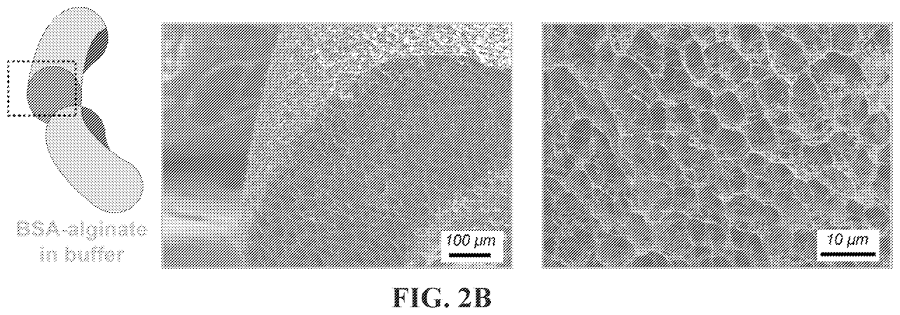

SEM imaging was used to examine the effects of an aqueous environment on the physical morphology of the BSA-alginate hydrogels. Following the initial crosslinking of BSA network for a varying amount of time, the soft protein gel was extruded into Ca²⁺ and kept under light for a total time of 30 min, to allow the competitive formation of both networks. Two samples were prepared for SEM imaging, one which was maintained in Ca²⁺ solution (top), and one that was exposed to Tris buffer solution for >30 minutes prior to lyophilization (bottom). The sample kept in Ca²⁺ solution exhibits a physical structure lacking noticeable pores and showing some larger holes (FIG. 2A). Extrusion into the Ca²⁺ solution during continued exposure to light allows for the simultaneous formation of the primary covalently bound protein network and secondary ion-triggered alginate network and stiffens the gel enough to enable it to survive the freeze-drying process required for the preparation of the SEM samples. The samples further incubated in Tris/EDTA buffer show a highly porous structure, with large vacancies, which are homogenously dispersed throughout the material (FIG. 2B). The material has a spongy conformation with pores throughout that are over 100 nm in size. Such large pores should be visible with laser scanning confocal microscopy, if also present before the freeze-drying step.

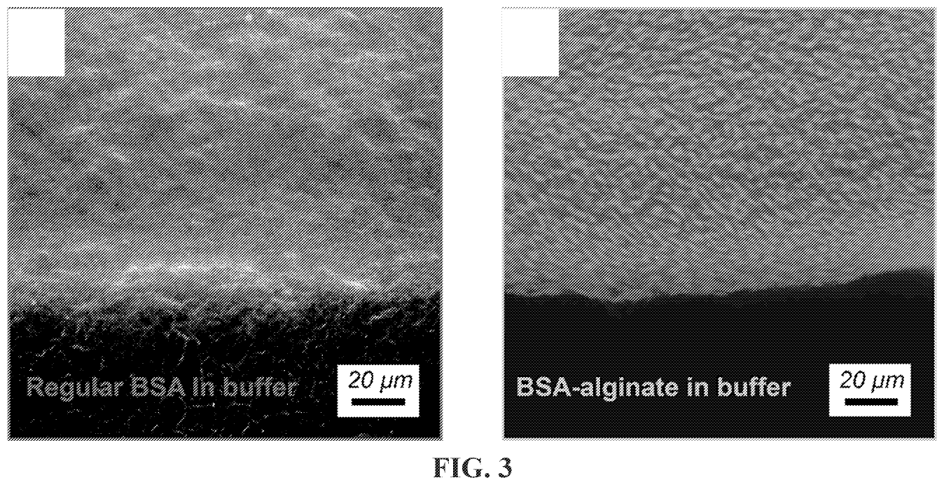

To assess the porosity of protein-polysaccharide gels compared to regular protein-based materials, they were first stained with rhodamine B, a thiol-reactive fluorescent dye. Confocal laser scanning microscopy was used to compare the physical properties of BSA-alginate and BSA hydrogel samples that were incubated in Tris buffer solution for at least 30 minutes. Unlike SEM, laser scanning microscopy allows for imaging of samples in an aqueous environment, eliminating the possibility of introducing pores or other physical defects during the freeze-drying and lyophilization processes. The BSA hydrogel sample appears uniform, lacking noticeable pores, as it is comprised of a single covalent network (FIG. 3 left panel).

Comparatively, the BSA-alginate specimen exhibits a homogeneous distribution with pores of 5±1 μm in diameter (FIG. 3 right panel). The difference in morphology may be explained by the diffusion of the secondary network from the BSA-alginate hydrogel, which leaves behind vacancies within the primary covalent network.

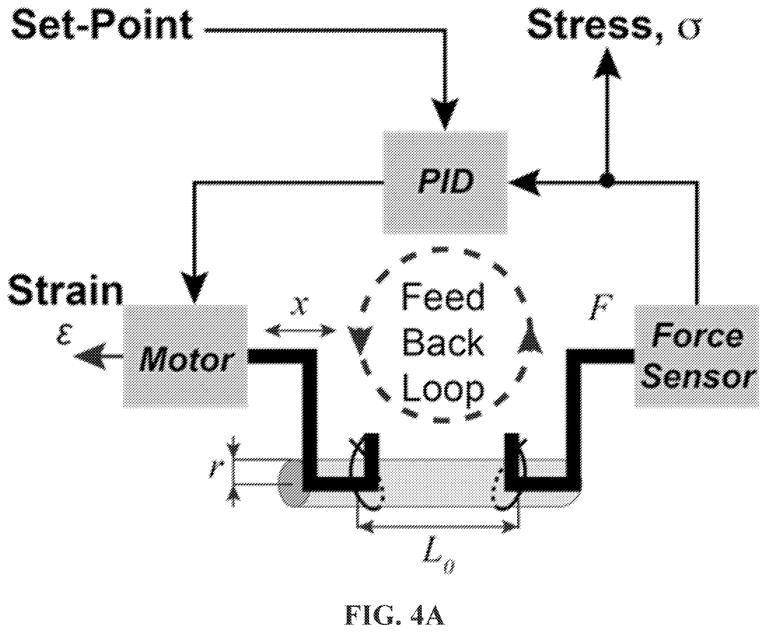

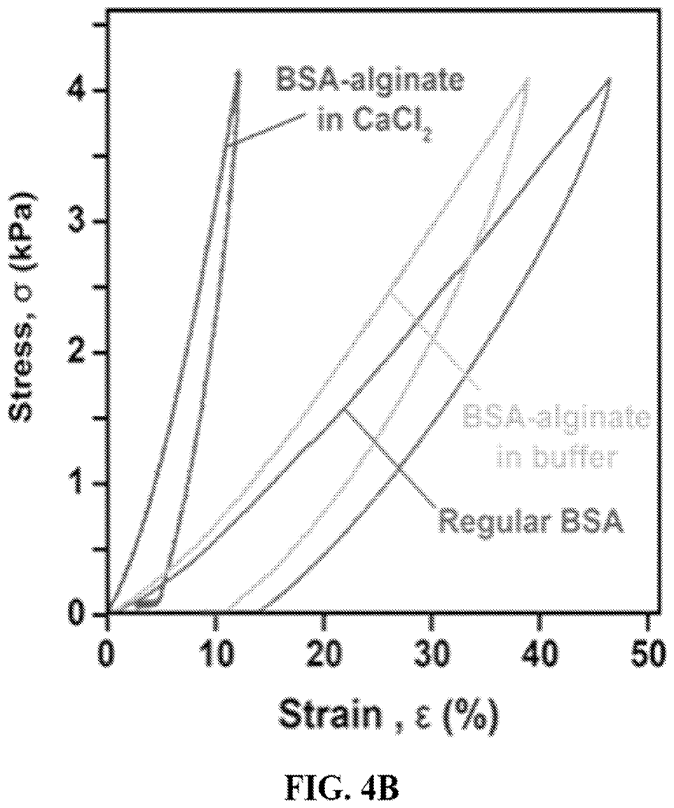

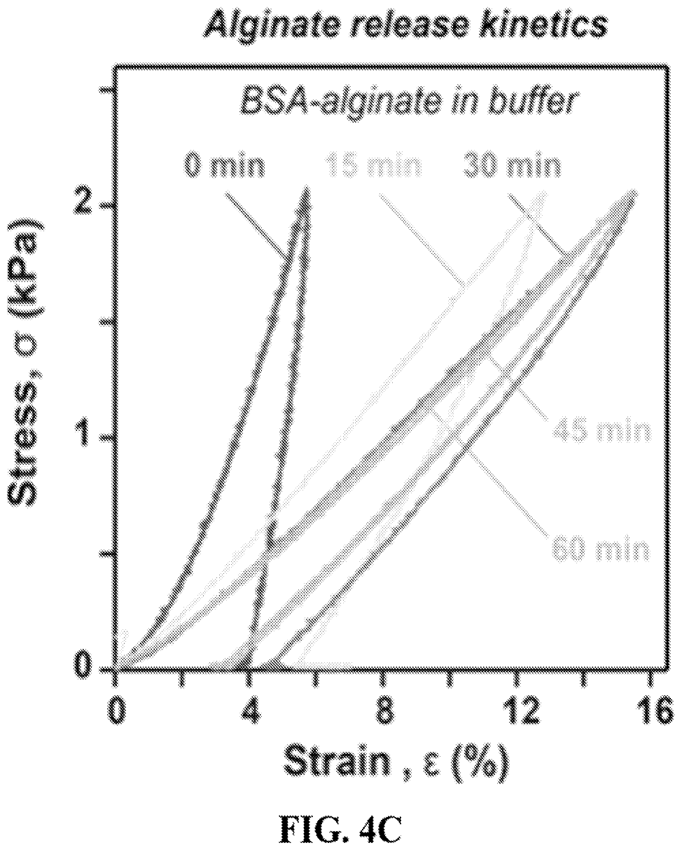

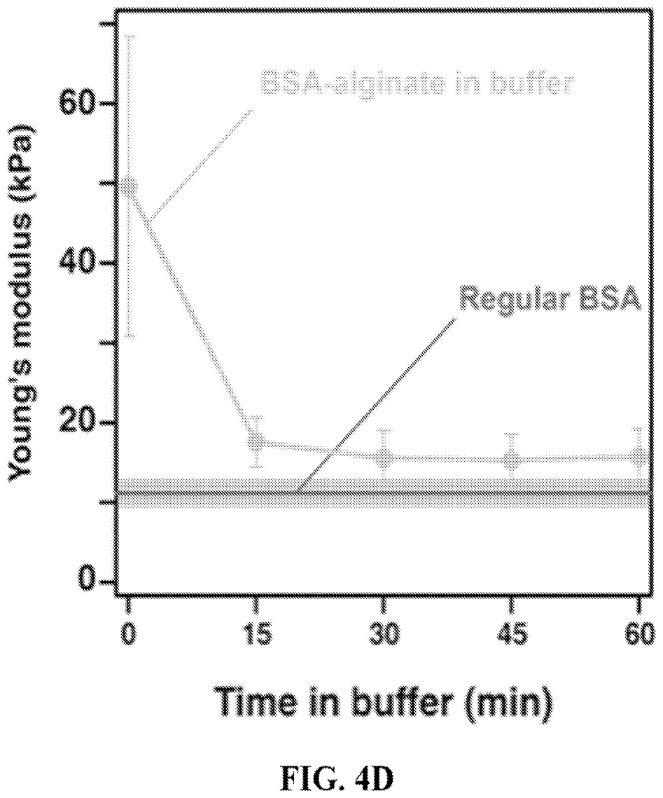

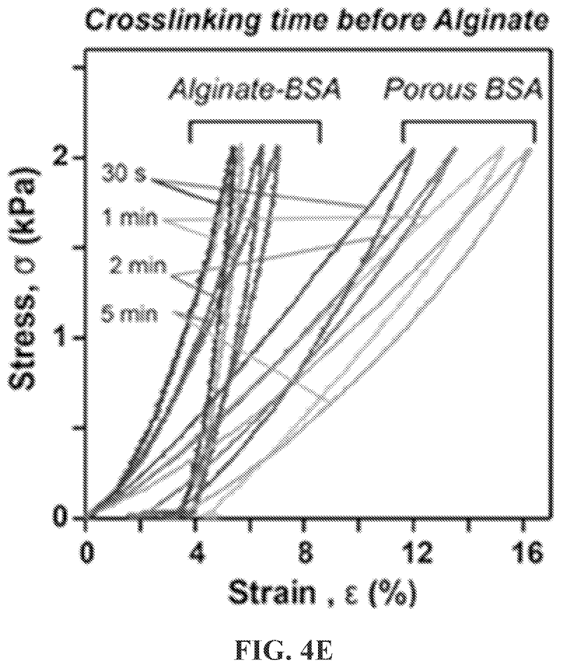

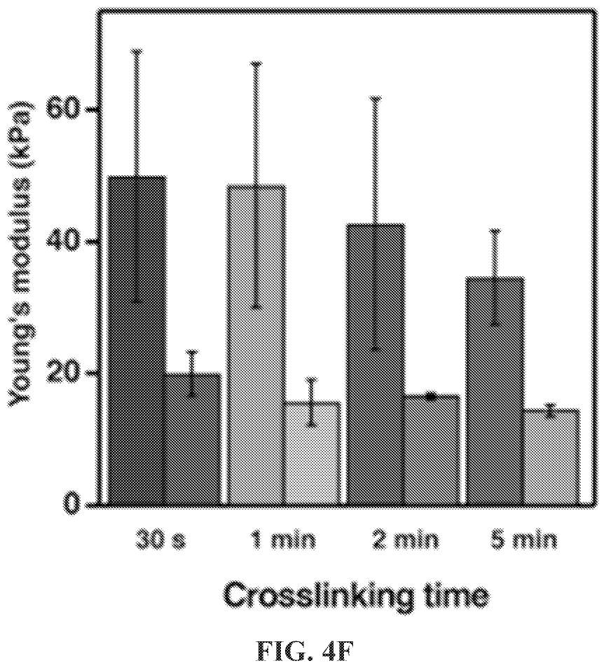

A significant drawback of currently used methods to produce protein-based porous materials is that they come at the expense of the primary gel structure. As protein-based materials are relatively soft, further weakening their structural integrity can compromise their stability and function. To test if the disclosed approach of forming the secondary network does not come at the expense of the primary backbone, force-clamp rheometry was used and compared the elasticity of the porous biomaterials obtained with the disclosed method against the same materials without large pores. First, cylindrical samples of standard and porous BSA hydrogels were prepared in polytetrafluoroethylene (PTFE) tubes with an inner diameter of 0.56 mm. The samples were maintained at room temperature, in CaCl₂ solution for the BSA-alginate samples, and Tris for the BSA samples, for ˜30 min. The materials were tethered with surgical thread to the two hooks on the force-clamp rheometer, attached to a voice coil motor and a force-sensor, respectively (FIG. 4A). A force-ramp protocol was then applied, stretching the samples to a peak force of 2 or 4 kPa at a rate of 40 Pa/s, while a PID-regulated feedback loop ensured that the applied force matched the set protocol (FIG. 4B). The data on applied force and corresponding change in gel length were used to generate stress-strain dependencies, in which the slope of the elastic region reports on the Young's modulus. The BSA-alginate hydrogels were initially stretched to a peak stress of 4 kPa in CaCl₂ solution, and relaxed back to zero force (FIG. 4B). After this initial stretch, the solution inside the chamber was changed to Tris or EDTA, and the gels were incubated for an additional 30 minutes before being stretched once more to 4 kPa. The measured Young's moduli for the same gel revealed a decrease in stiffness from 49±18 kPa in CaCl₂ solution to 13±3 kPa, after 30 minutes of incubation in Tris buffer solution. The change in mechanical response is due to the diffusion of the secondary ionic network outside the gel, as removing the calcium-alginate clusters leaves vacancies within the primary covalent network and increases the elasticity. For comparison, the pure-protein hydrogels were stretched to a peak stress of 4 kPa, as well, in a Tris buffer. Interestingly, the Young's modulus of the BSA hydrogels in Tris (11±2 kPa) was close to the stiffness of the porous BSA gels after removal of the calcium-alginate network. This resemblance suggests that introducing pores within the protein-polysaccharide hydrogel does not compromise the mechanical properties compared to a pure-protein network of 2 mM BSA that lacks pores.

The next step was to determine how the degradation of the calcium-alginate network from protein-polysaccharide hydrogels affects the mechanical properties of the remaining protein network over time. Four replicates of 2 mM BSA 2% (w/v) alginate hydrogel samples were prepared by exposing to light for 1 min, followed by immersion in Ca²⁺ solution under more light, for an additional 29 min. The mechanical response of the BSA-alginate hydrogels was measured first in Ca²⁺ solution and then monitored at 15 minute intervals over the course of an hour following transfer to tris buffer solution. The corresponding stress-strain curves (FIG. 4C) and calculated average Young's moduli (FIG. 4D) reveal a decrease in stiffness that stabilizes after about 30 minutes of incubation in Tris, at a specific point the Young's Modulus plateaus around ˜13 kPa and does not continue to decrease with continued exposure to Tris. Thus, it takes ˜30 minutes for the calcium-alginate network to degrade and diffuse from the primary network, resulting in a decrease in stiffness due to the introduction of pores. During the equilibration experiments, the samples were stretched at a rate of 40 Pa/s, but the peak stress was decreased from 4 kPa to 2 kPa, so that the same gel can be evaluated in 15 min intervals. An interesting finding is that the materials after alginate removal do not become weaker than the BSA control, which was cross-linked in the absence of the polysaccharide. In fact, the lowest Young's modulus measured is slightly higher than the Young's modulus of the control BSA, which might be due to some residual alginate molecules trapped inside the hydrogel structure.

The influence of crosslinking time for the primary network before inducing the parallel formation of the alginate secondary network was investigated (FIG. 4E and FIG. 4F). For this experiment the BSA-alginate mixture was first exposed to varying times, up to 30 min. The materials were then extruded in Ca²⁺ solution and continued the reaction, such that the total exposure time to light was 30 min. An interesting finding was that gels exposed for more than 5 min to light before immersion in Ca²⁺ solution were too brittle and broke immediately, suggesting that a complete crosslinking of the primary network followed by the growth of the secondary network compromises the integrity of the material and a immature primary network is needed for this approach. Second, the Young's moduli in Ca²⁺ solution showed a slow decrease with maturation time of the primary network, while the Young's moduli after removal of the primary network (Tris for 30 min) was constant from 1 min. These values suggest that the pore-inducing progression is an all-or-none process, and that a subtle kinetic competition between the formation of the primary and secondary networks drives pore formation.

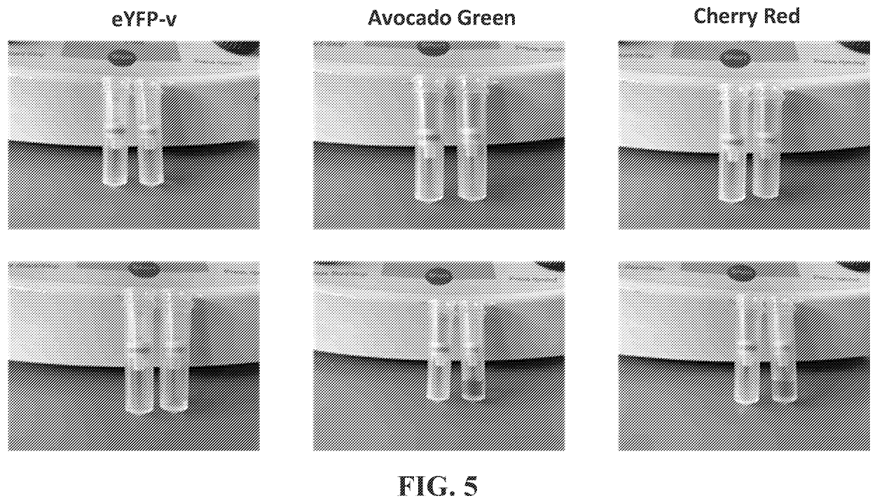

The change in porosity resulting from removing the calcium-alginate network from protein-polysaccharide hydrogels presents new transport opportunities through these materials. To assess the transfer properties of BSA biomaterials made with the disclosed method, BSA and BSA-alginate hydrogels were cast in a disk shape that fits regular plastic columns, used in filtration and separation experiments with lab centrifuges. These differences were explored by examining the ability of porous and nonporous 2 mM BSA networks, derived from the BSA-alginate and BSA hydrogels respectively, to pass through solutions of eYFP protein, and diluted food dyes (FIG. 5). First, disk-shaped hydrogels of each variety were prepared in a cylindrical shape (7.80 mm diameter, ˜3 mm height) and inserted into plastic columns with a ring on top, to prevent leakage of loaded solutions. Both hydrogel filters were first loaded with Tris buffer solution for at least 30 minutes. Next, the relative ability of each gel to pass test solutions was assessed by loading with equal volumes of each sample and recording the percent filtrate that passed through over the course of 1 minute, while spinning at 0.1 g. For each solution, the filter derived from the BSA-alginate hydrogel had a faster transfer rate than its pure-protein counterpart, with a greater volume of loaded solution passing through during the same duration of centrifugation. After 1 minute of centrifugation, about all the loaded volume of eYFP-vinculin solution passed through the BSA-alginate filter compared to 5% loaded volume that passed through the BSA filter. For the red and green dye solutions, the BSA-alginate filter passed close to 100% of the loaded volume after 1 minute of centrifugation. In contrast, its pure-protein counterpart passed under 1% of the loaded volume. This behavior can be explained by the greater porosity of the BSA-alginate filter, which allows for a faster transfer. Additionally, the concentration of eYFP filtrate that passed through the protein-polysaccharide-treated gel was consistently higher than that of the filtrate from the pure-protein filter. Compared to the loaded eYFP-vinculin concentration, the filtrates from the BSA-alginate and BSA filters had concentrations that were 61±15% and 28±9% of the original value, respectively.

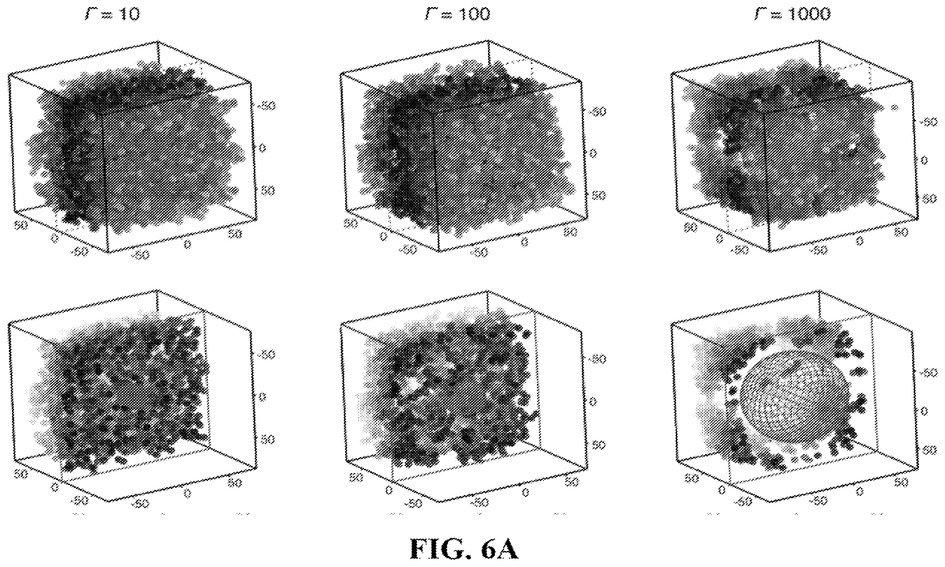

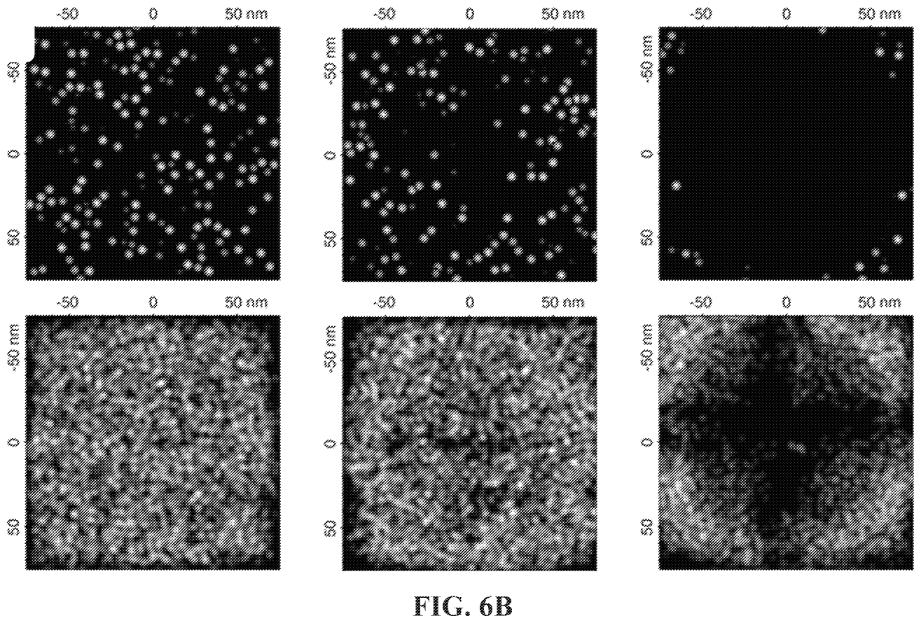

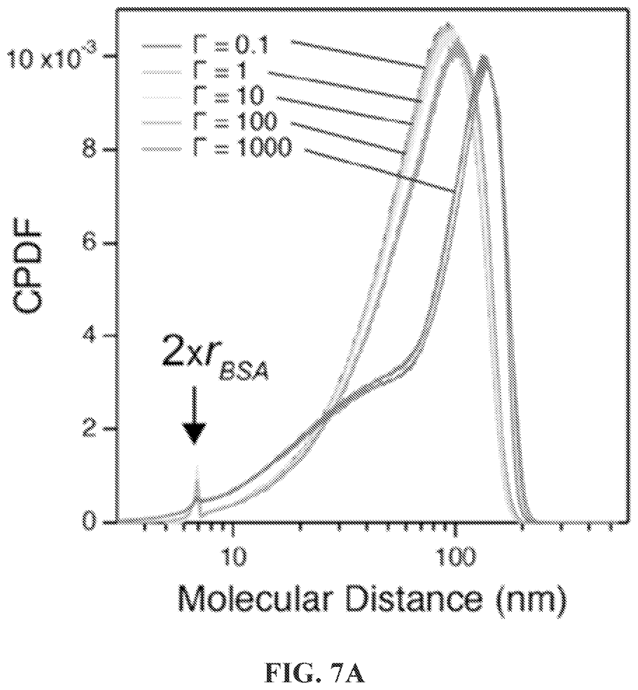

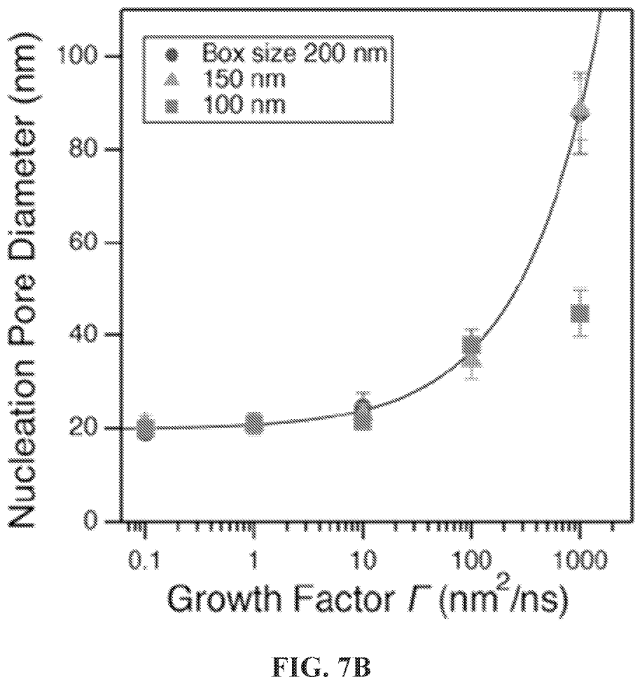

To visualize the formation of pores through the dynamic competition between cross-linking of protein molecules and protein-clusters and the nucleation of polysaccharides into growing polysaccharide-growing-centers, a model was used for cross-linking of protein hydrogels. In this model, the protein molecules start from a non-overlapping state, representing the BSA domains in solution. This approximation is valid, because BSA is a highly charged protein that is fully soluble at the considered concentration of 2 mM. As the protein molecules diffuse and collide, they form irreversible connections, which simulates the covalent bonds being formed between BSA molecules when the solution is exposed to light. A repulsive field exerts a force inversely proportional with the distance between the molecule/cluster from the center of the simulation volume, with a proportionality parameter Γ. This field at the center of the simulation volume represents the nucleation of the alginate into growing aggregates. While alginate nucleation was deemed to be instantaneous when measured at the seconds to minutes time-scales, in the simulation time-frame the parameter Γ is expected to have a finite value. To explore the range of values for the clustering rate, hydrogel unit volumes for 2 mM BSA were generated with different values for Γ and for three different box sizes (with a unit length of 100, 150 and 200 nm respectively) (FIG. 6A—top shows the 150 nm box size). The porosity of the cross-linked gels was evaluated by using a numerical method that determines the largest pore volume that can be inoculated inside the gel structure (FIG. 6A—bottom sphere and FIG. 7B). Furthermore, to predict the percolation size through the unit volume, the middle cross-section was inspected (FIG. 6B—top) and collapsed all the layers along a coordinate, obtaining a representative image of the pore connectivity within the sampled volume (FIG. 6B—bottom). The simulations indicate that the nucleation growth rate of alginate plays an important role in the formation of the central pore inside the simulation volume.

The porosity of a material can be described through the cumulative probability distribution function (CPDF) between the molecules inside a simulation volume. For the considered three volume sizes, CPDF shows a shift in the peak of the molecular distance with the nucleation speed from an average molecular distance normalized per box size of 0.61 to 0.87 for the considered range of growth factor values (FIG. 7A). As the model focuses on a single nucleation point located in the center of the simulation volume, a more important parameter is the size of the maximum pore (FIG. 7B). Using a numerical calculation of the maximum inscribed sphere, the maximum pore changes from ˜20 to ˜85 nm in the considered range of growth factor values. Note that the lower value for Γ=1000 and box size of 100 nm is due to geometry constraints for these conditions. From the change in pore size with the nucleation velocity, a scaling power law was determined.

While it is tempting to estimate a nucleation rate for the measured pore size of 5 μm, the simulations have some inherent limitations. The box size is limited in size by the simulation time. Currently, it is not feasible to have a simulation volume of a few microns, which would be large enough to simulate pore similar to those measured from microscopy images. Furthermore, if assumed that each collision is successful, while in reality only BSA molecules that collide with activated tyrosine amino acids in the vicinity form covalent bonds. As the success rate of this reaction is unknown, the effect of the nucleation factor will be underestimated in the simulation. Significant for this approach, however, is the fact that within the chosen simulation volumes and times, the process reaches equilibrium, as the pore sizes are independent of the simulation volume. Importantly though, the model captures the delicate balance between the nucleation rate of alginate and the cross-linking of protein hydrogels.

### Example 3

### Use of Porous Protein Structures

Networks of crosslinked protein were made using protein L. Protein L was engineered and expressed as octameric protein having 8 protein L molecules attached in tandem. This protein is referred to as protein L8.

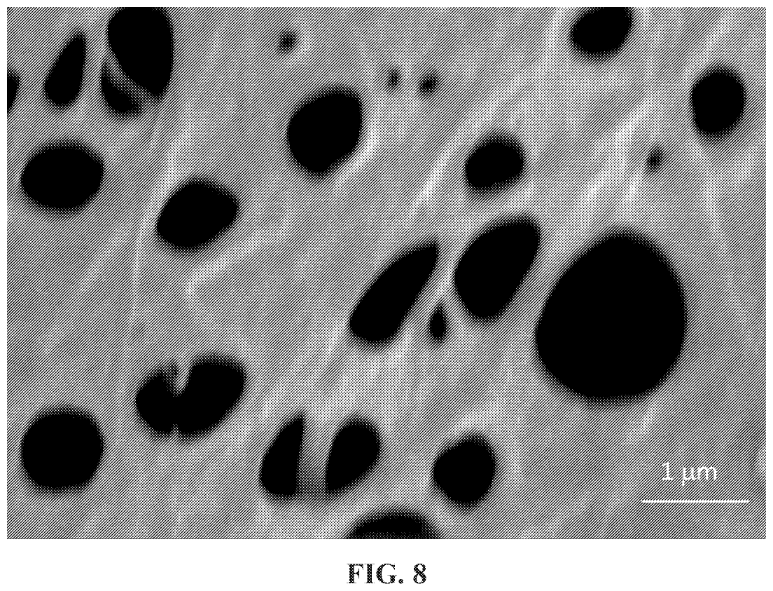

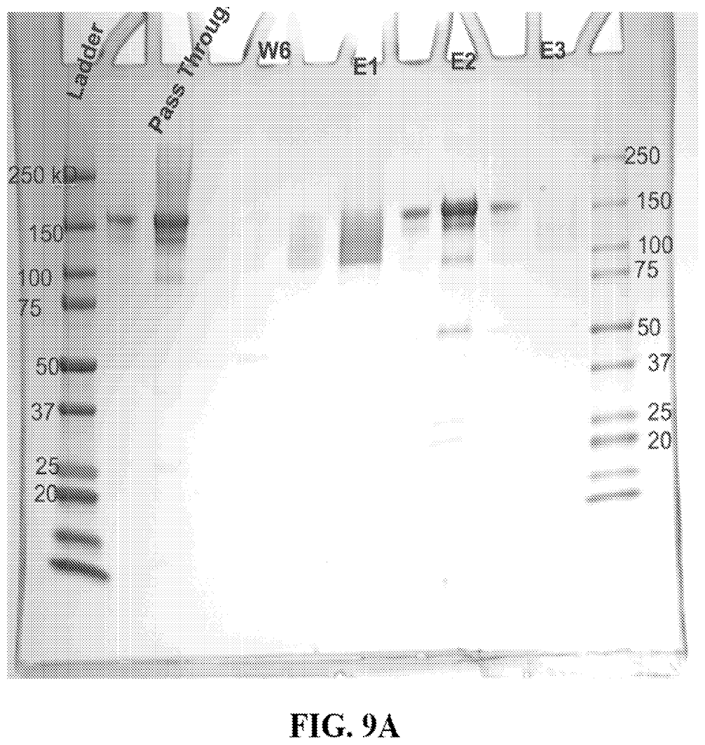

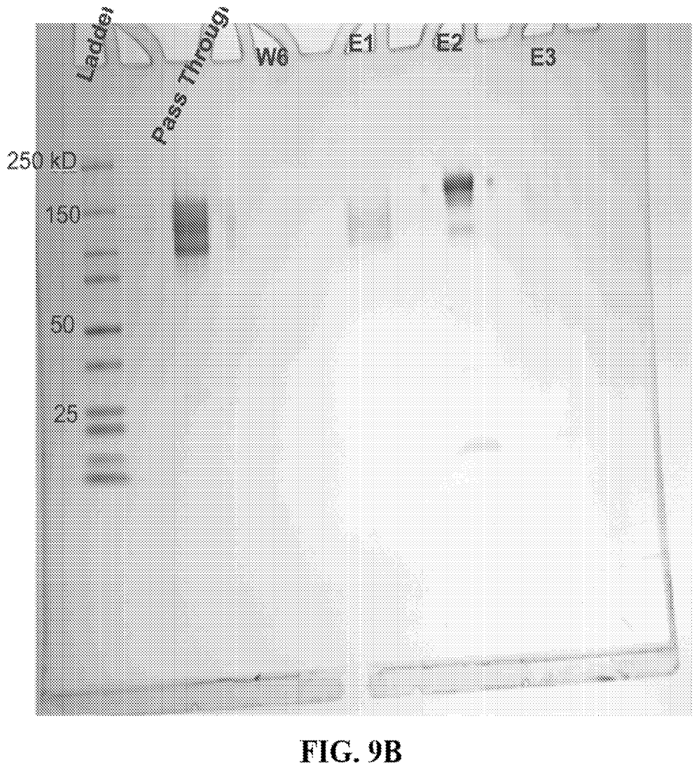

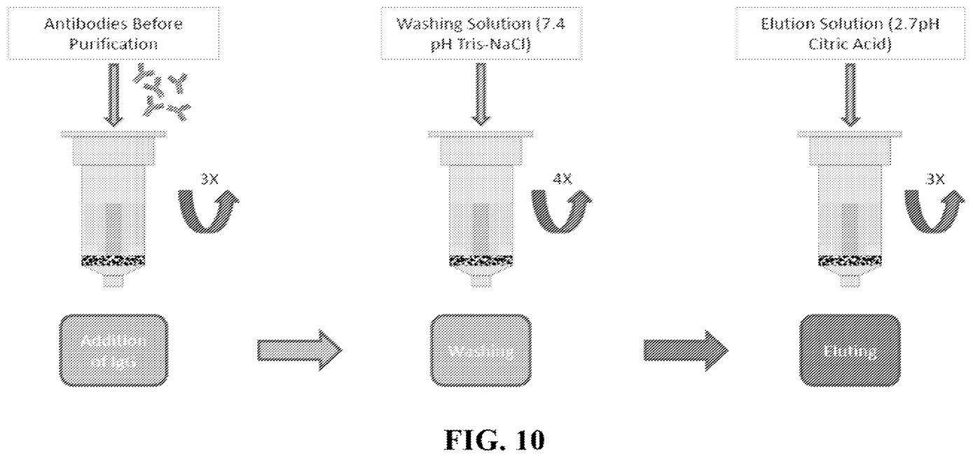

Networks of crosslinked protein L8 were made in a similar manner as was done in Examples 1 & 2 for BSA. An example image of a network of crosslinked protein L8 can be seen in FIG. 8. Furthermore, crosslinked networks of protein L8 were used in columns for purification of antibodies (FIG. 9A, FIG. 9B, and FIG. 10). As seen in FIG. 9A and FIG. 9B, the network of crosslinked protein L8 made via the disclosed methods showed a higher binding capacity than the network made from protein L8 without alginate. These results are further detailed below in Table 1.

TABLE 1
Details of gels shown in FIG. 9A and FIG. 9B

| | FIG. 9A | FIG. 9B |
|---|---|---|
| Sample tested | 1.5 mM protein L8 | 1.0 mM protein L8 |
| | with 0.5% (w/v) alginate | with no alginate |
| Total binding (overall | Total binding of 1047.32 ug/ml | Total binding of 689 ug/ml |
| result) | | |
| Lane 1 | Ladder | Ladder |
| Lane 2 | Blank/no sample loaded | Blank/no sample loaded. |
| | This lane shows bands because | |
| | sample leaked over from lane 3 | |
| | (pass through) | |
| Lane 3 | Pass through | Pass through |
| | (non-adsorbed antibody) | (non-adsorbed antibody) |
| Lane 4 | Blank/no sample | Blank/no sample |
| 5 | W6/Wash | W6/Wash |
| | No bands demonstrate that non- | No bands demonstrate that non- |
| | specifically adsorbed antibody was | specifically adsorbed antibody was |
| | washed. | washed. |
| 6 | Blank/no sample loaded. | Blank/no sample loaded. |
| | This lane shows bands because | |
| | sample leaked over from lane 7 | |
| | (E1) | |
| 7 | El/First round of antibody elution | El/First round of antibody elution |
| 8 | Blank/no sample loaded. | Blank/no sample loaded. |
| | This lane shows bands because | |
| | sample leaked over from lane 9 | |
| | (E2) | |
| 9 | E2/Second round of antibody | E2/Second round of antibody elution |
| | elution | |
| 10 | Blank/no sample loaded. | Blank/no sample loaded. |
| | This lane shows bands because | |
| | sample leaked over from lane 9 | |
| | (E2) | |
| 11 | E3/Third round of antibody elution | E3/Third round of antibody elution |
| 12 | Ladder | Blank/no sample loaded. |

Similar porous compositions will also be made of protein A4, protein A5, and protein G8 and used in columns for purification of analytes. In addition, porous compositions including albumin covalently attached to an antibody will be made and used in columns for purification.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A composition comprising: a network of crosslinked protein, the network of crosslinked protein including a plurality of pores, each pore having a diameter of greater than 500 nm, wherein the network of crosslinked protein has a Young's modulus of greater than 1 kPa.

Clause 2. The composition of clause 1, wherein the protein comprises an albumin, an antibody binding protein, an antibody, a fragment thereof, or a combination thereof.

Clause 3. The composition of clause 2, wherein the antibody binding protein is selected from the group consisting of protein G, protein L, protein A, protein M, and a combination thereof.

Clause 4. The composition of any one of clauses 1-3, wherein the protein has a molecular weight of about 20 kDa to about 100 kDa.

Clause 5. The composition of any one of clauses 1-4, wherein the protein comprises a protein having at least 3 tyrosine residues.

Clause 6. The composition of any one of clauses 1-5, wherein the protein is crosslinked by enzymatic crosslinking, photocrosslinking, thermal crosslinking, a crosslinking agent, or a combination thereof.

Clause 7. The composition of clause 6, wherein the crosslinking agent is a light activated crosslinking agent, a redox-activated crosslinking agent, or a combination thereof.

Clause 8. The composition of any one of clauses 1-7, wherein the protein is crosslinked through tyrosine residues.

Clause 9. The composition of any one of clauses 1-8, further comprising a binding ligand associated with the network of crosslinked protein.

Clause 10. The composition of any one of clauses 1-9, wherein the network of crosslinked protein is a hydrogel.

Clause 11. A composition comprising: a network of crosslinked protein, the network of crosslinked protein including a plurality of pores, wherein the protein comprises an albumin, an antibody binding protein, an antibody, a fragment thereof, or a combination thereof, each pore has a diameter of greater than 1 μm, and the network of crosslinked protein has a Young's modulus of greater than 1 kPa and less than 100 kPa.

Clause 12. A method of making a porous network of crosslinked protein, the method comprising: a. combining a protein and a polysaccharide in a solvent to provide a mixture; b. crosslinking the protein to provide a partial first network; c. crosslinking the partial first network and the polysaccharide, wherein crosslinking the partial first network provides a first network and crosslinking the polysaccharide provides a second network dispersed in the first network; and d. adding the first network and the second network to a solvent, wherein the solvent removes the second network from the first network to provide a porous network of crosslinked protein.

Clause 13. The method of clause 12, wherein the second network forms spherical structures dispersed in the first network.

Clause 14. The method of clause 12 or 13, wherein step c is performed no more than 10 minutes after step b.

Clause 15. The method of any one of clauses 12-14, wherein the partial first network formed in step b is no more than 50% crosslinked compared to the first network.

Clause 16. The method of any one of clauses 12-15, wherein the protein comprises an albumin, an antibody binding protein, an antibody, a fragment thereof, or a combination thereof.

Clause 17. The method of any one of clauses 12-16, wherein the polysaccharide comprises alginate, cellulose, carrageenan, pectin, sulfated polysaccharides, or a combination thereof.

Clause 18. The method of any one of clauses 12-17, wherein the polysaccharide has a molecular weight of about 10 kDa to about 100 kDa, the protein has a molecular weight of about 20 kDa to about 100 kDa, or a combination thereof.

Clause 19. The method of any one of clauses 12-18, wherein the polysaccharide is alginate.

Clause 20. The method of any one of clauses 12-19, wherein the first network is covalently crosslinked and the second network is electrostatically crosslinked.

Clause 21. A method of separating an analyte from a sample, the method comprising: contacting the sample with a chromatography matrix, the chromatography matrix comprising the composition of any one of clauses 1-11; and separating the analyte from the sample.

Clause 22. The method of clause 21, wherein the chromatography matrix is capable of passing through molecules having a hydrodynamic diameter of greater than or equal to 100 nm as measured by dynamic light scattering.

Clause 23. The method of clause 21 or 22, wherein the analyte comprises a protein.

SEQUENCES

(SEQ ID NO: 1) Sequence (protein A-domain B4)8
MRGSHHHHHHGSADNKFNKEQQNAFYEILHLPNLNEEQRNGFIQSLKDDPSQSANLLA
EAKKLNDAGSADNKFNKEQQNAFYEILHLPNLNEEQRNGFIQSLKDDPSQSANLLAEAK
KLNDAGSADNKFNKEQQNAFYEILHLPNLNEEQRNGFIQSLKDDPSQSANLLAEAKKLN
DAGSADNKFNKEQQNAFYEILHLPNLNEEQRNGFIQSLKDDPSQSANLLAEAKKLNDAG
SADNKFNKEQQNAFYEILHLPNLNEEQRNGFIQSLKDDPSQSANLLAEAKKLNDAGSAD
NKFNKEQQNAFYEILHLPNLNEEQRNGFIQSLKDDPSQSANLLAEAKKLNDAGSADNKF
NKEQQNAFYEILHLPNLNEEQRNGFIQSLKDDPSQSANLLAEAKKLNDAGSADNKFNKE
QQNAFYEILHLPNLNEEQRNGFIQSLKDDPSQSANLLAEAKKLNDAGS
(SEQ ID NO: 2) Sequence (protein A-domain B5)8
MRGSHHHHHHGSQAPKADNKFNKEQQNAFYEILHLPNLTEEQRNGFIQSLKDDPSVSKE
ILAEAKKLNDARSQAPKADNKFNKEQQNAFYEILHLPNLTEEQRNGFIQSLKDDPSVSKE
ILAEAKKLNDARSQAPKADNKFNKEQQNAFYEILHLPNLTEEQRNGFIQSLKDDPSVSKE
ILAEAKKLNDARSQAPKADNKFNKEQQNAFYEILHLPNLTEEQRNGFIQSLKDDPSVSKE
ILAEAKKLNDARSQAPKADNKFNKEQQNAFYEILHLPNLTEEQRNGFIQSLKDDPSVSKE
ILAEAKKLNDARSQAPKADNKFNKEQQNAFYEILHLPNLTEEQRNGFIQSLKDDPSVSKE
ILAEAKKLNDARSQAPKADNKFNKEQQNAFYEILHLPNLTEEQRNGFIQSLKDDPSVSKE
ILAEAKKLNDARSQAPKADNKFNKEQQNAFYEILHLPNLTEEQRNGFIQSLKDDPSVSKE
ILAEAKKLNDARS
(SEQ ID NO: 3) Sequence (protein G-domain B1)8
MRGSHHHHHHGSMEYKLILNGKTLKGETTTEAVDAATAEKVFKQYANDNGVDGEWT
YDDATKTFTVTERSMEYKLILNGKTLKGETTTEAVDAATAEKVFKQYANDNGVDGEW
TYDDATKTFTVTERSMEYKLILNGKTLKGETTTEAVDAATAEKVFKQYANDNGVDGE
WTYDDATKTFTVTERSMEYKLILNGKTLKGETTTEAVDAATAEKVFKQYANDNGVDG
EWTYDDATKTFTVTERSMEYKLILNGKTLKGETTTEAVDAATAEKVFKQYANDNGVD
GEWTYDDATKTFTVTERSMEYKLILNGKTLKGETTTEAVDAATAEKVFKQYANDNGV
DGEWTYDDATKTFTVTERSMEYKLILNGKTLKGETTTEAVDAATAEKVFKQYANDNG
VDGEWTYDDATKTFTVTERSMEYKLILNGKTLKGETTTEAVDAATAEKVFKQYANDN
GVDGEWTYDDATKTFTVTERS
(SEQ ID NO: 4) Sequence (protein L-domain B1)8
MRGSHHHHHHGSMEEVTIKANLIFANGSTQTAEFKGTFEKATSEAYAYADTLKKDNGE
WTVDVADKGYTLNIKFRSMEEVTIKANLIFANGSTQTAEFKGTFEKATSEAYAYADTLK
KDNGEWTVDVADKGYTLNIKFRSMEEVTIKANLIFANGSTQTAEFKGTFEKATSEAYAY
ADTLKKDNGEWTVDVADKGYTLNIKFRSMEEVTIKANLIFANGSTQTAEFKGTFEKATS
EAYAYADTLKKDNGEWTVDVADKGYTLNIKFRSMEEVTIKANLIFANGSTQTAEFKGT
FEKATSEAYAYADTLKKDNGEWTVDVADKGYTLNIKFRSMEEVTIKANLIFANGSTQT
AEFKGTFEKATSEAYAYADTLKKDNGEWTVDVADKGYTLNIKFRSMEEVTIKANLIFA
NGSTQTAEFKGTFEKATSEAYAYADTLKKDNGEWTVDVADKGYTLNIKFRSMEEVTIK
ANLIFANGSTQTAEFKGTFEKATSEAYAYADTLKKDNGEWTVDVADKGYTLNIKFRS

What is claimed is:

1. A composition comprising:
a network of crosslinked protein, the network of crosslinked protein including a plurality of pores, each pore having a diameter of greater than 500 nm, wherein the network of crosslinked protein has a Young's modulus of greater than 1 kPa.

2. The composition of claim 1, wherein the protein comprises an albumin, an antibody binding protein, an antibody, a fragment thereof, or a combination thereof.

3. The composition of claim 2, wherein the antibody binding protein is selected from the group consisting of protein G, protein L, protein A, protein M, and a combination thereof.

4. The composition of claim 1, wherein the protein has a molecular weight of about 20 kDa to about 100 kDa.

5. The composition of claim 1, wherein the protein comprises a protein having at least 3 tyrosine residues.

6. The composition of claim 1, wherein the protein is crosslinked by enzymatic crosslinking, photocrosslinking, thermal crosslinking, a crosslinking agent, or a combination thereof.

7. The composition of claim 6, wherein the crosslinking agent is a light activated crosslinking agent, a redox-activated crosslinking agent, or a combination thereof.

8. The composition of claim 1, wherein the protein is crosslinked through tyrosine residues.

9. The composition of claim 1, further comprising a binding ligand associated with the network of crosslinked protein.

10. The composition of claim 1, wherein the network of crosslinked protein is a hydrogel.

11. A composition comprising:
a network of crosslinked protein, the network of crosslinked protein including a plurality of pores, wherein:
the protein comprises an albumin, an antibody binding protein, an antibody, a fragment thereof, or a combination thereof,
each pore has a diameter of greater than 1 μm, and
the network of crosslinked protein has a Young's modulus of greater than 1 kPa and less than 100 kPa.

12. A method of making a porous network of crosslinked protein, the method comprising:
a. combining a protein and a polysaccharide in a solvent to provide a mixture;
b. crosslinking the protein to provide a partial first network;
c. crosslinking the partial first network and the polysaccharide, wherein crosslinking the partial first network provides a first network and crosslinking the polysaccharide provides a second network dispersed in the first network; and
d. adding the first network and the second network to a solvent, wherein the solvent removes the second network from the first network to provide a porous network of crosslinked protein.

13. The method of claim 12, wherein the second network forms spherical structures dispersed in the first network.

14. The method of claim 12, wherein step c is performed no more than 10 minutes after step b.

15. The method of claim 12, wherein the partial first network formed in step b is no more than 50% crosslinked compared to the first network.

16. The method of claim 12, wherein the protein comprises an albumin, an antibody binding protein, an antibody, a fragment thereof, or a combination thereof.

17. The method of claim 12, wherein the polysaccharide comprises alginate, cellulose, carrageenan, pectin, sulfated polysaccharides, or a combination thereof.

18. The method of claim 12, wherein the polysaccharide has a molecular weight of about 10 kDa to about 100 kDa, the protein has a molecular weight of about 20 kDa to about 100 kDa, or a combination thereof.

19. The method of claim 12, wherein the polysaccharide is alginate.

20. The method of claim 12, wherein the first network is covalently crosslinked and the second network is electrostatically crosslinked.

21. A method of separating an analyte from a sample, the method comprising:
contacting the sample with a chromatography matrix, the chromatography matrix comprising the composition of claim 1; and
separating the analyte from the sample.

22. The method of claim 21, wherein the chromatography matrix is capable of passing through molecules having a hydrodynamic diameter of greater than or equal to 100 nm as measured by dynamic light scattering.

23. The method of claim 21, wherein the analyte comprises a protein.


SEQUENCE LISTING (4 sequences)

SEQ ID NO: 1 - protein, 460 aa, synthetic construct
MRGSHHHHHHGSADNKFNKEQQNAFYEILHLPNLNEEQRNGFIQSLKDDPSQSANLLAEAKKLNDAGSADNKFNKEQQNAFYEILHLPNLNEEQRNGFIQSLKDDPSQSANLLAEAKKLNDAGSADNKFNKEQQNAFYEILHLPNLNEEQRNGFIQSLKDDPSQSANLLAEAKKLNDAGSADNKFNKEQQNAFYEILHLPNLNEEQRNGFIQSLKDDPSQSANLLAEAKKLNDAGSADNKFNKEQQNAFYEILHLPNLNEEQRNGFIQSLKDDPSQSANLLAEAKKLNDAGSADNKFNKEQQNAFYEILHLPNLNEEQRNGFIQSLKDDPSQSANLLAEAKKLNDAGSADNKFNKEQQNAFYEILHLPNLNEEQRNGFIQSLKDDPSQSANLLAEAKKLNDAGSADNKFNKEQQNAFYEILHLPNLNEEQRNGFIQSLKDDPSQSANLLAEAKKLNDAGS

SEQ ID NO: 2 - protein, 492 aa, synthetic construct
MRGSHHHHHHGSQAPKADNKFNKEQQNAFYEILHLPNLTEEQRNGFIQSLKDDPSVSKEILAEAKKLNDARSQAPKADNKFNKEQQNAFYEILHLPNLTEEQRNGFIQSLKDDPSVSKEILAEAKKLNDARSQAPKADNKFNKEQQNAFYEILHLPNLTEEQRNGFIQSLKDDPSVSKEILAEAKKLNDARSQAPKADNKFNKEQQNAFYEILHLPNLTEEQRNGFIQSLKDDPSVSKEILAEAKKLNDARSQAPKADNKFNKEQQNAFYEILHLPNLTEEQRNGFIQSLKDDPSVSKEILAEAKKLNDARSQAPKADNKFNKEQQNAFYEILHLPNLTEEQRNGFIQSLKDDPSVSKEILAEAKKLNDARSQAPKADNKFNKEQQNAFYEILHLPNLTEEQRNGFIQSLKDDPSVSKEILAEAKKLNDARSQAPKADNKFNKEQQNAFYEILHLPNLTEEQRNGFIQSLKDDPSVSKEILAEAKKLNDARS

SEQ ID NO: 3 - protein, 476 aa, synthetic construct
MRGSHHHHHHGSMEYKLILNGKTLKGETTTEAVDAATAEKVFKQYANDNGVDGEWTYDDATKTFTVTERSMEYKLILNGKTLKGETTTEAVDAATAEKVFKQYANDNGVDGEWTYDDATKTFTVTERSMEYKLILNGKTLKGETTTEAVDAATAEKVFKQYANDNGVDGEWTYDDATKTFTVTERSMEYKLILNGKTLKGETTTEAVDAATAEKVFKQYANDNGVDGEWTYDDATKTFTVTERSMEYKLILNGKTLKGETTTEAVDAATAEKVFKQYANDNGVDGEWTYDDATKTFTVTERSMEYKLILNGKTLKGETTTEAVDAATAEKVFKQYANDNGVDGEWTYDDATKTFTVTERSMEYKLILNGKTLKGETTTEAVDAATAEKVFKQYANDNGVDGEWTYDDATKTFTVTERSMEYKLILNGKTLKGETTTEAVDAATAEKVFKQYANDNGVDGEWTYDDATKTFTVTERS

SEQ ID NO: 4 - protein, 524 aa, synthetic construct
MRGSHHHHHHGSMEEVTIKANLIFANGSTQTAEFKGTFEKATSEAYAYADTLKKDNGEWTVDVADKGYTLNIKFRSMEEVTIKANLIFANGSTQTAEFKGTFEKATSEAYAYADTLKKDNGEWTVDVADKGYTLNIKFRSMEEVTIKANLIFANGSTQTAEFKGTFEKATSEAYAYADTLKKDNGEWTVDVADKGYTLNIKFRSMEEVTIKANLIFANGSTQTAEFKGTFEKATSEAYAYADTLKKDNGEWTVDVADKGYTLNIKFRSMEEVTIKANLIFANGSTQTAEFKGTFEKATSEAYAYADTLKKDNGEWTVDVADKGYTLNIKFRSMEEVTIKANLIFANGSTQTAEFKGTFEKATSEAYAYADTLKKDNGEWTVDVADKGYTLNIKFRSMEEVTIKANLIFANGSTQTAEFKGTFEKATSEAYAYADTLKKDNGEWTVDVADKGYTLNIKFRSMEEVTIKANLIFANGSTQTAEFKGTFEKATSEAYAYADTLKKDNGEWTVDVADKGYTLNIKFRS